(19) United States
(12) Patent Application Publication
(10) Pub. No.: US 2008/0096863 A1
(43) Pub. Date: Apr. 24, 2008

(54) Stable pharmaceutical compositions of calcium channel blocker and an ACE inhibitor

(75) Inventors: Rahul Maheshchandra Nagaria, Dist. Gandhinagar (IN); Abhay Anantrao Vaidya, Dist. Gandhinagar (IN); Srinivasa Ramachandra Murthy Achanta, Dist. Gandhinagar (IN); Sunil Sadanand Nadkarni, Dist. Gandhinagar (IN)

Correspondence Address: DARBY & DARBY P.C., P.O. BOX 770, Church Street Station, New York, NY, 10008-0770, US

(73) Assignee: Torrent Pharmaceuticals Limited, Dist. Gandhinagar (IN)

(21) Appl. No.: 11/636,536

(22) Filed: Dec. 11, 2006

(30) Foreign Application Priority Data
Oct. 19, 2006 (IN) 1732/MUM/2006

(51) Int. Cl. A61K 31/554; A61K 31/455; A61K 31/401; A61K 31/407

(52) U.S. Cl. 514/211.07; 514/355; 514/412; 514/423

(57) ABSTRACT

The present invention relates to a stable pharmaceutical composition of a combination of amlodipine and an ACE inhibitor; wherein the two active ingredients are not physically separated and the composition has a pH of more than 6.0. It also relates to a process for preparation, and a method for using such a composition.

### FIELD OF THE INVENTION

The invention relates to a stable pharmaceutical composition of a combination of amlodipine and an angiotensin converting enzyme (ACE) inhibitor, a process for preparation, and a method for using such composition.

### BACKGROUND OF THE INVENTION

Calcium channel blockers and angiotensin converting enzyme (ACE) inhibitors are widely used for the treatment of hypertension and related disorders.

Amlodipine, a calcium channel blocker, and its salts are disclosed in U.S. Pat. No. 4,572,909. Further, the besylate salt of amlodipine is disclosed in U.S. Pat. No. 4,879,303. It is known that amlodipine is hygroscopic in nature and may degrade in presence of moisture. One of the major routes of degradation is known to be pH-dependent catalytic oxidative process. The 3-ethyl 5-methyl 2-[(2-aminoethoxy) methyl]-4-(2-chlorophenyl)-6-methylpyridine-3,5-dicarboxylate (“Impurity D” of Formula (I)) is typically a cause of stability concern in case of amlodipine formulation.

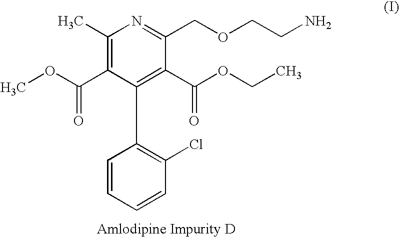

Ramipril, an angiotensin converting enzyme (ACE) inhibitor, and its physiologically acceptable salts are disclosed in U.S. Pat. No. 5,061,722. Chemically, it is designated as (2S,3aS,6aS)-1{-(S)-N-([(S)-1-carboxy-3-phenylpropyl]alanyl}octahydro-cyclopenta pyrrole-2-carboxylic acid 1-ethyl ester and is used for the treatment of hypertension, heart failure, and nephropathy. Ramipril is susceptible to degradation by hydrolysis to ramipril diacid [(2S,3aS,6aS)-1-[(S)-2-[[(S)-1-carboxy-3-phenylpropyl]amino]propanoyl]octahydrocyclopenta [b]pyrrole-2-carboxylic acid (“Impurity E” of Formula (II)). This is an active metabolite of ramipril and therefore may not necessarily require control in the formulation. It is also susceptible to cyclization to ramipril diketopiperazide [ethyl (2S)-2-[(3S,5aS,8aS,9aS)-3-methyl-1,4-dioxodecahydro-2H-cyclopenta[4,5]pyrrolo[1,2-a]pyrazin-2-yl]-4-phenylbutanoate (“Impurity D” of Formula (III)). These impurities are defined in European Pharmacopoeia.

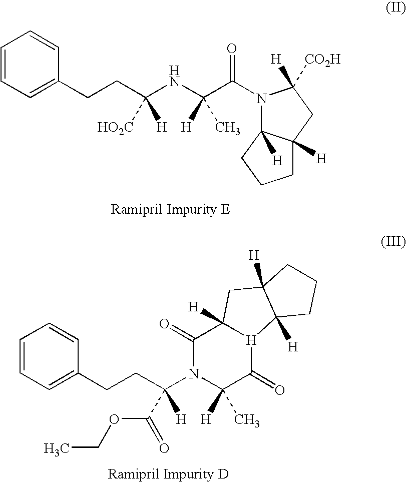

Benazepril, benazeprilat, and their pharmaceutically acceptable salts are disclosed in U.S. Pat. No. 4,410,520, along with pharmaceutically acceptable dosage forms thereof, dosage ranges and suitable routes of administration therewith, and uses therefor.

Combination of calcium channel blocker and ACE inhibitor has been known to provide synergistic effect in the treatment of cardiovascular disorders such as hypertension. It would be desirable to prepare fixed dose pharmaceutical combination of a calcium channel blocker such as amlodipine and an ACE inhibitor such as ramipril or benazepril, to increase compliance for patients taking such combination. However, preparation of fixed dose stable pharmaceutical compositions of such a combination of amlodipine and ramipril or amlodipine and benazepril are difficult to prepare due to the incompatibility of the active ingredients. Ramipril may show more stability related problems during formulation than that of benazepril. Ramipril shows a tendency to be unstable in pharmaceutical formulations, depending on the excipients used, the manufacturing process and storage; high temperature, humidity and compression are the factors that determine the stability of the formulations with ramipril.

U.S. Pat. No. 6,162,802 claims a pharmaceutical composition consisting of amlodipine and benazepril such that the two drugs are physically separated from each other. The patent specification discloses that physical separation of amlodipine and benazepril is necessary as they are incompatible substances. Physical separation of the two drugs has been shown to be achieved in a number of ways; coating pellets of one active, before incorporating into a tablet of the other, separately coating pellets of each active and then filling in a capsule, coating pellets of one active and filling in a capsule with powder of the other active, microencapsulating each active separately in order to ensure that the two drugs do not come in contact and then blending together for use in a tablet or capsule, use of a dual or multiple compartment transdermal device, etc.

The BR 00/03282 patent application assigned to Libbs Pharmaceuticals Ltd. discloses the pharmaceutical association of amlodipine and ramipril for the treatment of arterial hypertension and prevention of other cardiovascular diseases such as myocardial infarction, cerebrovascular disorders and cardiac and renal insufficiency. The patent specification discloses the composition in which amlodipine and ramipril are physically isolated. The patent specification discloses the capsule dosage form with two kinds of coated granules (amlodipine granules and ramipril granules), the capsule dosage form with two coated tablets (one coated tablet of amlodipine and one coated tablet of ramipril) and the coated tablet containing amlodipine and ramipril separated by an inert layer.

The WO 06/85208 patent application assigned to Ranbaxy Laboratories Limited discloses stable solid dosage forms of amlodipine and benazepril, wherein the dosage form contains two components. The first component comprising amlodipine or pharmaceutically acceptable salts thereof and is substantially free of dicalcium phosphate and the second component comprising benazepril or pharmaceutically acceptable salts thereof. The dosage form has less than about 0.2% concentration (w/w) of impurity D after three months at 40° C. and 75% RH.

The WO 06/97943 patent application assigned to Lupin Limited discloses the stable pharmaceutical compositions of amlodipine, or its pharmaceutically acceptable salt and benazepril, or its pharmaceutically acceptable salt, characterized in that the two drugs are not physically separated from each other. The stabilization is achieved by using excipients other than alkali and alkaline earth metal carbonates and phosphates and those excipients, which increase the pH of microenvironment above 5. The application also states that amlodipine : benazepril combination may be incompatible with alkali and alkaline earth metal carbonates and phosphates. The patent specification also states that excipients, which increase the pH of the blend above 5, resulted in color change of the preformulation blend. These excipients include meglumine, alkali and alkaline earth metal carbonates or phosphates.

It has been surprisingly found that a stable composition of a combination of amlodipine and an ACE inhibitor can be prepared using basifying agents without the need of physical separation of the two active ingredients and without compromising the stability.

### SUMMARY OF THE INVENTION

In one aspect, the invention discloses a stable pharmaceutical composition comprising
(a) a calcium channel blocker;
(b) an ACE inhibitor;
(c) one or more basifying agents; and
(d) optionally one or more pharmaceutically acceptable excipients,
   wherein the calcium channel blocker and the ACE inhibitor are not physically separated.

In another aspect, the invention discloses a stable pharmaceutical composition comprising:
(i) 1-15% by weight calcium channel blocker,
(ii) 1-15% by weight of an ACE inhibitor
(iii) 1-10% by weight hydroxypropyl methylcellulose,
(iv) 30-90% by weight microcrystalline cellulose, and
(v) 0.1-10% by weight magnesium carbonate;
   wherein the calcium channel blocker and the ACE inhibitor are not physically separated.

In further aspect, the invention discloses a method for treatment of hypertension, wherein the method comprises administering a patient in need thereof a stable pharmaceutical composition comprising
(a) a calcium channel blocker;
(b) an ACE inhibitor;
(c) one or more basifying agents; and
(d) optionally one or more pharmaceutically acceptable excipients,
   wherein the calcium channel blocker and the ACE inhibitor are not physically separated.

### DETAILED DESCRIPTION OF THE INVENTION

The term “stable pharmaceutical composition” as used herein refers to the composition of amlodipine and an ACE inhibitor wherein the impurities do not exceed the regulatory requirement of country of interest. For example, stable pharmaceutical composition refers to compositions comprising amlodipine impurity D less than 0.2% w/w and ramipril impurity D less than 0.2% w/w when stored at 30° C. and 65% relative humidity (RH) for a period of one month.

The term “amlodipine” as used herein includes amlodipine free base, or pharmaceutically acceptable acid addition salts thereof or mixtures thereof. It also covers anhydrous form, hydrous form, different crystalline forms, amorphous form, enantiomers of amlodipine or pharmaceutically acceptable acid addition salts thereof or mixtures thereof. Amlodipine may be present in an amount ranging from 1% to 15% by weight of the composition.

The term “ACE inhibitor” as used herein includes angiotensin converting enzyme inhibitor selected from ramipril, benazepril, captopril, enalapril, quinapril, perindopril, lisinopril, fosinopril, trandolapril, moexipril or pharmaceutically acceptable salts thereof. ACE inhibitor may be present in an amount ranging from 1% to 15% by weight of the composition.

The term “ramipril” as used herein includes ramipril free base, ramiprilat, or pharmaceutically acceptable salts of ramipril or mixtures thereof.

The term “benazepril” as used herein includes benazepril free base, benazeprilat or pharmaceutically acceptable salts of benazepril or mixtures thereof.

Calcium channel blockers are compounds which work by blocking voltage-sensitive calcium channels in the heart and in the blood vessels. This prevents calcium levels from increasing as much in the cells when stimulated, leading to less contraction. This decreases total peripheral resistance by dilating the blood vessels, and decreases cardiac output by lowering the force of contraction. Because resistance and output drop, so does blood pressure.

With low blood pressure, the heart does not have to work as hard; this can ease problems with cardiomyopathy and coronary disease. Unlike with beta-blockers, the heart is still responsive to sympathetic nervous system stimulation, so blood pressure can be maintained more effectively.

The term “calcium channel blockers” as used herein includes (1) Dihydropyridine calcium channel blockers including (a) Amlodipine (Norvasc), (b) Felodipine (Plendil), (c) Nicardipine (Cardene, Carden SR), (d) Nifedipine (Procardia, Adalat), (e) Nimodipine (Nimotop), (f) Nisoldipine (Sular), (g) Nitrendipine (Cardif, Nitrepin), (h) Lacidipine (Motens), and (i) Lercanidipine (Zanidip); (2) Phenylalkylamine calcium channel blockers including (a) Verapamil (Calan, Isoptin), and (b) Gallopamil (D600); (3) Benzothiazepine calcium channel blockers including Diltiazem (Cardizem); and (4) other calcium channel blocker such as Menthol (mint oil). The calcium channel blocker(s) may be present in an amount ranging from 1% to 15% by weight of the composition.

The term “basifying agent” as used herein refers to compounds which has pH of more than 6.0 and are capable of increasing the pH of the formulation to more than 6.0. Preferably, the basifying agent comprises alkali or alkaline earth metal carbonates, phosphates, oxides or hydroxides. The alkali or alkaline earth metal carbonate comprises sodium carbonate, sodium bicarbonate, calcium carbonate or magnesium carbonate. The alkali or alkaline earth metal phosphate comprises sodium phosphate, disodium phosphate, trisodium phosphate, dibasic calcium phosphate or calcium phosphate anhydrous. The alkali or alkaline earth metal oxide comprises magnesium oxide or aluminium oxide. The basifying agent may be present in an amount ranging from 0.1% to 10% by weight of the composition. It would be apparent to a person skilled in the art that some basifying agents may be used as diluents in higher concentration.

The term “core” as used herein includes sugar globules, inert non-pareil seeds, inert beads, inert pellets, granules, flakes, tablets and such like.

The term “not physically separated” is intended to mean that calcium channel blocker and the ACE inhibitor are present together in a single unit or a single phase in the composition. Physical separation is described in U.S. Pat. No. 6,162,802 [column 3, line 50 to 58]. This may be accomplished in any of the myriad ways known in the art, such as bi-layered tablets, coated pellets of one agent incorporated into a tablet of the other, separately coated pellets of each agent in a capsule or tablet, coated pellets of one agent in capsule together with powder of the other agent, each agent microencapsulated separately and then blended together for use in a tablet or capsule, use of a dual or multiple compartment transdermal device, etc. Due to the incompatibility, combination products of the two agents in an injectable solution are not really acceptable. For example, amlodipine and ACE inhibitor as disclosed in the compositions of U.S. Pat. No. 6,162,802 and Brazilian patent application No. 00/03282 are physically separated.

The pharmaceutical compositions as described herein may comprise one or more pharmaceutically acceptable excipients selected from diluent, disintegrant, binder, film forming agent, lubricant, and such like.

Diluent may be selected from powdered cellulose, microcrystalline cellulose, silicified microcrystalline cellulose, starch, dextrose; sugars such as lactose or sucrose; sugar alcohols such as mannitol, sorbitol or erythritol; or mixtures thereof. The diluent may be present in an amount ranging from 1% to 90% by weight of the composition.

Disintegrant may be selected from croscarmellose sodium, sodium starch glycolate, pregelatinized starch, sodium carboxymethyl cellulose, microcrystalline cellulose, cross-linked polyvinylpyrrolidone or mixtures thereof. The disintegrant may be present in an amount ranging from 1% to 10% by weight of the composition.

Binder may be selected from hydroxyethyl cellulose, hydroxypropyl cellulose, hydroxypropyl methylcellulose, carbomers, dextrin, ethyl cellulose, methylcellulose, shellac, zein, gelatin, polymethacrylates, polyvinyl pyrrolidone, pregelatinized starch, sodium alginate, gums, synthetic resins, and such like. The binder may be present in an amount ranging from 0.1% to 10% by weight of the composition.

Film forming agent may be selected from hydroxypropyl methylcellulose, polyvinylpyrrolidone, gelatin, hydroxypropyl cellulose, polyethylene oxide, hydroxyethyl cellulose, sodium alginate and such like. The film forming agent may be used in seal coat, drug coat, separating coat, film coat and such like.

Lubricant/glidant may be selected from talc, metallic stearates such as magnesium stearate, calcium stearate, zinc stearate; colloidal silicon dioxide, finely divided silicon dioxide, stearic acid, hydrogenated vegetable oil, glyceryl palmitostearate, glyceryl monostearate, glyceryl behenate, polyethylene glycols, starch, sodium stearyl fumarate, mineral oil, magnesium trisilicate; or mixtures thereof. The lubricant/glidant may be present in an amount ranging from 0.1% to 10% by weight of the composition.

One embodiment discloses a stable pharmaceutical composition comprising
(a) a calcium channel blocker;
(b) an ACE inhibitor;
(c) one or more basifying agents; and
(d) optionally one or more pharmaceutically acceptable excipients,
   wherein amlodipine and ramipril are not physically separated from each other and the composition has a pH of more than 6.0.

Another embodiment discloses a stable pharmaceutical composition comprising
(a) amlodipine;
(b) ramipril/benazepril;
(c) one or more basifying agents; and
(d) optionally one or more pharmaceutically acceptable excipients,
   wherein amlodipine and ramipril are not physically separated from each other and the composition has a pH of more than 6.0.

Another embodiment discloses stable pharmaceutical composition comprising:
(i) 1-30% by weight amlodipine,
(ii) 1-30% by weight ramipril/benazepril,
(iii) 1-20% by weight hydroxypropyl methylcellulose,
(iv) 10-90% by weight microcrystalline cellulose, and
(v) 0.1-10% by weight magnesium carbonate;
   wherein amlodipine and ramipril are not physically separated and the composition has a pH of more than 6.0.

Another embodiment discloses a process for preparing a stable pharmaceutical composition, wherein the process comprises the steps of:
mixing a calcium channel blocker, an ACE inhibitor, one or more basifying agents and optionally one or more pharmaceutically acceptable excipients;
granulating the mixture;
drying the granules;
optionally mixing the granules with one or more pharmaceutically acceptable excipients, and
filling the granules into capsules.

Another embodiment discloses a process for preparing a stable pharmaceutical composition, wherein the process comprises the steps of:
mixing a calcium channel blocker, an ACE inhibitor, one or more basifying agents and optionally one or more pharmaceutically acceptable excipients,
granulating the mixture to obtain wet mass,
extruding the wet mass to obtain the pellets,
optionally mixing the pellets with one or more pharmaceutically acceptable excipients, and
filling the pellets into capsules.

Still another embodiment discloses a process for preparing a stable pharmaceutical composition, wherein the process comprises the steps of:
mixing a calcium channel blocker, one or more basifying agents and optionally one or more pharmaceutically acceptable excipients,
granulating the mixture to obtain wet mass,
extruding the wet mass to obtain the pellets,
coating the pellets with an ACE inhibitor, and
filling the coated pellets into capsules.

Still another embodiment discloses a process for preparing a stable pharmaceutical composition, wherein the process comprises the steps of:
mixing an ACE inhibitor, one or more basifying agents and optionally one or more pharmaceutically acceptable excipients,
granulating the mixture to obtain wet mass,
extruding the wet mass to obtain the pellets,
coating the pellets with a calcium channel blocker,
optionally mixing the pellets with one or more pharmaceutically acceptable excipients, and
filling the coated pellets into capsules.

Further embodiment discloses a process for preparing a stable pharmaceutical composition, wherein the process comprises the steps of:
mixing a calcium channel blocker and an ACE inhibitor with one or more basifying agents and optionally one or more pharmaceutically acceptable excipients,
coating the mixture on a core,
optionally mixing the coated core with one or more pharmaceutically acceptable excipients, and
filling the coated cores into capsules.

The pharmaceutical compositions as described herein may be illustrated by the following examples which are not to be construed as limiting the scope of the invention:

### COMPARATIVE EXAMPLE

| | |
|---|---|
| Ingredients | Qty. in mg |

Seal-coating of sugar globules

| | |
|---|---|
| Sugar Globules | 65.00 |
| Hydroxypropyl methylcellulose (HPMC) | 5.00 |
| Talc | 2.00 |
| Purified Water | q.s. |
| Total | 72.00 |

Drug coating of seal-coated globules

| | |
|---|---|
| Seal-coated sugar globules | 72.00 |
| Ramipril | 5.00 |
| Amlodipine besylate | 6.93 |
| Hydroxypropyl methylcellulose (HPMC) | 10.17 |
| Sodium Phosphate | 1.00 |
| Purified Water | q.s. |
| Total | 95.10 |

Film coating of drug coated pellets

| | |
|---|---|
| Drug Coated Pellets | 95.10 |
| Hydroxypropyl methylcellulose (HPMC) | 8.00 |
| Talc | 2.90 |
| Purified Water | q.s. |
| Total | 106.00 |

PROCEDURE: HPMC and talc were dispersed in water and the suspension was coated on sugar globules to provide a seal coat. Ramipril, amlodipine besylate, HPMC and sodium phosphate were mixed and dispersed in water and the suspension was coated on seal-coated sugar globules to obtain pellets. The pellets were coated with a suspension of HPMC and talc in water, dried and filled into the capsule of appropriate size. The pH of the pellets was 5.20.

### EXAMPLE 1

| Ingredients | Qty. in mg |
|---|---|
| Ramipril | 5.00 |
| Amlodipine besylate | 6.93 |
| Microcrystalline cellulose | 62.49 |
| Magnesium carbonate | 0.55 |
| Color | 0.03 |
| Purified water | q.s. |
| Total | 75.00 |

PROCEDURE: Ramipril, amlodipine besylate, microcrystalline cellulose, magnesium carbonate and color were sifted and mixed in a rapid mixer granulator. The mixture was granulated with purified water to obtain a wet mass. The wet mass was passed through an extruder and the extrudates obtained were spheronized to obtain pellets of required size. The pellets were dried and filled into the capsule of appropriate size. The pH of the pellets was 8.40.

### EXAMPLE 2

| Ingredients | Qty. in mg |
|---|---|
| Ramipril | 5.00 |
| Amlodipine besylate | 6.93 |
| Microcrystalline cellulose | 56.72 |
| Magnesium carbonate | 1.35 |
| Purified water | q.s. |
| Hydroxypropyl methylcellulose (HPMC) | 5.20 |
| Talc | 1.29 |
| Color | 0.01 |
| Purified water | q.s. |
| Total | 76.50 |

PROCEDURE: Ramipril, amlodipine besylate, microcrystalline cellulose and magnesium carbonate were sifted and mixed in rapid mixer granulator. The mixture was granulated with purified water and the granules obtained were dried and sized. A coating suspension was prepared by dispersing HPMC, talc and color in purified water and the granules were coated by spraying the suspension over the granules. Coated granules were filled into the capsules of appropriate size. The pH of the granules of was 8.40.

### EXAMPLE 3

| Ingredients | Qty. in mg |
|---|---|
| Ramipril | 5.00 |
| Amlodipine besylate | 6.93 |
| Microcrystalline cellulose | 57.25 |
| Magnesium carbonate | 0.82 |
| Purified water | q.s. |
| Hydroxypropyl methylcellulose (HPMC) | 20.00 |
| Talc | 5.00 |
| Purified water | q.s. |
| Total | 95.00 |

PROCEDURE: Ramipril, amlodipine besylate, microcrystalline cellulose and magnesium carbonate were sifted and mixed in rapid mixer granulator. The mixture was granulated with purified water and the granules obtained were dried and sized. A coating suspension was prepared by dispersing HPMC and talc in purified water and the granules were coated by spraying the suspension over the granules. Coated granules were filled into the capsules of appropriate size.

### EXAMPLE 4

| | |
|---|---|
| Ingredients | Qty/Cap(mg) |

Amlodipine spheres

| | |
|---|---|
| Amlodipine besylate | 6.93 |
| Dibasic calcium phosphate | 5.00 |
| Microcrystalline cellulose | 60.57 |
| Magnesium carbonate | 2.50 |
| Purified water | q.s. |
| Total | 75.00 |

Ramipril coating of Amlodipine spheres

| | |
|---|---|
| Ramipril | 5 |
| Magnesium oxide | 2.5 |
| Hydroxypropyl methylcellulose (HPMC) | 10 |
| Talc | 7.50 |
| Purified Water | q.s. |
| Total | 25.00 |

Coating of Drug coated spheres

| | |
|---|---|
| Hydroxypropyl methylcellulose (HPMC) | 20.00 |
| Talc | 10.00 |
| Purified Water | q.s. |
| Total | 130.00 |

PROCEDURE: Amlodipine besylate, dibasic calcium phosphate, microcrystalline cellulose and magnesium carbonate were sifted and mixed in a rapid mixer granulator. The mixture was granulated with purified water to obtain a wet mass. The wet mass was passed through an extruder and the extrudates obtained were spheronized to obtain spheres of required size. Ramipril, HPMC and talc were mixed and dispersed in water and the suspension was coated on amlodipine spheres to obtain pellets. The pellets were dried and filled into the capsule of appropriate size. The pH of the pellets was 9.80.

### EXAMPLE 5

| Ingredients | Qty. in mg |
|---|---|
| Benazepril | 5.00 |
| Amlodipine besylate | 6.93 |
| Microcrystalline cellulose | 56.72 |
| Magnesium carbonate | 1.35 |
| Purified water | q.s. |
| Hydroxypropyl methylcellulose (HPMC) | 5.20 |
| Talc | 1.30 |
| Purified water | q.s. |
| Total | 76.50 |

PROCEDURE: Benazepril, amlodipine besylate, microcrystalline cellulose and magnesium carbonate are sifted and mixed in rapid mixer granulator. The mixture is granulated with purified water and the granules obtained are dried and sized. A coating suspension is prepared by dispersing HPMC and talc in purified water and the granules are coated by spraying the suspension on the granules. Coated granules are filled into the capsule of appropriate size.

### EXAMPLE 6

| | |
|---|---|
| Ingredients | Qty. in mg |

Seal-coating of sugar globules

| | |
|---|---|
| Sugar Globules | 65.00 |
| Hydroxypropyl methylcellulose (HPMC) | 5.00 |
| Talc | 2.00 |
| Purified Water | q.s. |
| Total | 72.00 |

Drug coating of seal-coated globules

| | |
|---|---|
| Seal-coated sugar globules | 72.00 |
| Ramipril | 5.00 |
| Amlodipine besylate | 6.93 |
| Hydroxypropyl cellulose (HPC) | 10.17 |
| Magnesium carbonate | 1.00 |
| Purified Water | q.s. |
| Total | 95.10 |

Film coating of drug coated pellets

| | |
|---|---|
| Drug Coated Pellets | 95.10 |
| Hydroxypropyl methylcellulose (HPMC) | 8.00 |
| Talc | 2.90 |
| Purified Water | q.s. |
| Total | 106.00 |

PROCEDURE: HPMC and talc are dispersed in water and the suspension is coated on sugar globules to provide a seal coat. Ramipril, amlodipine besylate, HPC and magnesium carbonate are mixed and dispersed in water and the suspension is coated on seal-coated sugar globules to obtain pellets. The pellets are coated with a suspension of HPMC and talc in water, dried and filled into the capsule of appropriate size. The pH of the pellets is expected to be more than 6.

The compositions were subjected to stability studies at 30° C. and 65% relative humidity and the level of impurities were measured at regular interval. The impurities were measured by High Performance Liquid Chromatography (HPLC) using the stationary and the mobile phase system and conditions as below:

| | |
|---|---|
| Mobile phase | A: Buffer |
| | B: Acetonitrile |
| Column | C18, Waters 250 mm × 4.6 mm; 5μ |
| Flow | 1.0 ml/min |
| Detection | At 210 nm |
| Oven Temp. | 50° C. |
| Injection Volume | 20 μl |
| Run time | 120 Minutes. |
| Diluent | Buffer: Acetonitrile (50:50) |

The buffer may be prepared by dissolving 1.36 gm of potassium dihydrogen phosphate in 1000 ml water. 1 gm octane sulphonic acid sodium salt is added to the solution and sonicated. The pH is adjusted to 2.5±0.05 with orthophosphoric acid (10% v/v).

Gradient Program:

| Time(min) | Mobile Phase A | Mobile Phase B |
|---|---|---|
| 0.01 | 70 | 30 |
| 30.00 | 70 | 30 |
| 50.00 | 60 | 40 |
| 70.00 | 50 | 50 |
| 80.00 | 50 | 50 |
| 90.00 | 40 | 60 |
| 100.00 | 40 | 60 |
| 105.00 | 70 | 30 |
| 120.00 | 70 | 30 |

The standard solution was prepared by making a solution containing Ramipril Impurity D, Ramipril Impurity E and Amlodipine Impurity D at a concentration of 2.5 ppm in diluent. The test solution was prepared by dissolving the contents of the capsules in diluent to get a final concentration of 500 ppm of Ramipril and Amlodipine. The standard and the test solution were injected separately and the chromatograms were recorded. The level of impurities in the test was calculated by comparing with the standard chromatogram.

The result of the stability studies for Examples 2, 3, 4 and Comparative Example are summarized in Table 1:

TABLE 1
Stability studies for Examples 2, 3, 4 and Comparative Example

| | Example 2 | | | Example 3 | | Example 4 | |
|---|---|---|---|---|---|---|---|
| | Condition | | | | | | |
| | | 30° C./ | | | 30° C./ | | 30° C./ |
| Impurity | Ambient | 65% RH | | Ambient | 65% RH | Ambient | 65% RH |
| (% w/w) | Initial | 1M | 2M | Initial | 2M | Initial | 2 M |
| Amlodipine | 0.08 | 0.10 | 0.10 | 0.12 | 0.15 | ND* | 0.12 |
| Impurity D | | | | | | | |
| Ramipril | 0.13 | 0.16 | 0.12 | ND* | ND* | 0.14 | ND* |
| Impurity D | | | | | | | |
| Ramipril | 0.04 | 0.59 | 0.58 | 0.26 | 0.60 | — | 0.81 |
| Impurity E | | | | | | | |

[*ND = not detectable or below the level of quantification]

We claim:

1) A stable pharmaceutical composition comprising
(a) a calcium channel blocker;
(b) an ACE inhibitor;
(c) one or more basifying agents; and.
(d) optionally one or more pharmaceutically acceptable excipients,
wherein the calcium channel blocker and the ACE inhibitor are not physically separated.

2) The composition according to claim 1, wherein the calcium channel blocker comprises a compound selected from the group consisting of amlodipine, felodipine, nicardipine, nifedipine, nimodipine, nisoldipine, nitrendipine, lacidipine, lercanidipine, verapamil, gallopamil, diltiazem, menthol, pharmaceutically acceptable salts thereof, and combinations thereof.

3) The composition according to claim 2, wherein the calcium channel blocker is amlodipine.

4) The composition according to claim 1, wherein the ACE inhibitor comprises a compound selected from the group consisting of ramipril, benazepril, captopril, enalapril, quinapril, perindopril, lisinopril, fosinopril, trandolapril, moexipril, pharmaceutically acceptable salts thereof, and combinations thereof.

5) The composition according to claim 4, wherein the ACE inhibitor is ramipril or benazepril.

6) The composition according to claim 1, wherein the composition has a pH of more than 6.0.

7) The composition according to claim 6, wherein the composition has a pH in the range of 7 to 8.5.

8) The composition according to claim 1, wherein the basifying agent comprises alkali or alkaline earth metal carbonates, phosphates, oxides or hydroxides, and combinations thereof.

9) The composition according to claim 8, wherein the alkali or alkaline earth metal carbonate comprises sodium carbonate, sodium bicarbonate, calcium carbonate or magnesium carbonate, and combinations thereof.

10) The composition according to claim 8, wherein the alkali or alkaline earth metal phosphate comprises sodium phosphate, disodium phosphate, trisodium phosphate, dibasic calcium phosphate or calcium phosphate anhydrous, and combinations thereof.

11) The composition according to claim 8, wherein the alkali or alkaline earth metal oxide comprises magnesium oxide or aluminum oxide, and combinations thereof.

12) The composition according to claim 1, wherein the pharmaceutically acceptable excipients comprise one or more diluents, disintegrants, binders, film forming agents or lubricants, and combinations thereof.

13) The composition according to claim 1, wherein the composition is in the form of a tablet or a capsule.

14) A process of preparing a stable pharmaceutical composition according to claim 1, wherein the process comprises:
(i) mixing the calcium channel blocker, an ACE inhibitor, one or more basifying agents and optionally one or more pharmaceutically acceptable excipients;
(ii) granulating the mixture of step (i);
(iii) drying the granules of step (ii);
(iv) optionally mixing the granules of step (iii) with one or more pharmaceutically acceptable excipients; and
(v) filling the granules of step (iii) or product of step (iv) into capsules.

15) A process of preparing a stable pharmaceutical composition according to claim 1, wherein the process comprises:
(i) mixing the calcium channel blocker, an ACE inhibitor, one or more basifying agents and optionally one or more pharmaceutically acceptable excipients;
(ii) granulating the mixture of step (i) to obtain wet mass;
(iii) extruding the wet mass to obtain pellets;
(iv) optionally mixing the pellets of step (iii) with one or more pharmaceutically acceptable excipients; and
(v) filling the granules of step (iii) or product of step (iv) into capsules.

16) A process of preparing a stable pharmaceutical composition according to claim 1, wherein the process comprises:
(i) mixing the calcium channel blocker, an ACE inhibitor, one or more basifying agents and optionally one or more pharmaceutically acceptable excipients;
(ii) coating the mixture of step (i) on a core;
(iii) optionally mixing the coated core of step (ii) with one or more pharmaceutically acceptable excipients; and
(iv) filling the product of step (ii) into capsules.

17) A process of preparation of a stable pharmaceutical composition according to claim 1, wherein the process comprises:
(i) mixing the calcium channel blocker, one or more basifying agents and optionally one or more pharmaceutically acceptable excipients;
(ii) granulating the mixture of step (i) to obtain wet mass;
(iii) extruding the wet mass to obtain pellets;
(iv) coating the pellets with an ACE inhibitor;
(v) optionally mixing the pellets of step (iv) with one or more pharmaceutically acceptable excipients; and
(vi) filling the pellets of step (iv) or product of step (v) into capsules.

18) A process of preparation of a stable pharmaceutical composition according to claim 1, wherein the process comprises:
(i) mixing an ACE inhibitor, one or more basifying agents and optionally one or more pharmaceutically acceptable excipients;
(ii) granulating the mixture of step (i) to obtain wet mass;
(iii) extruding the wet mass to obtain pellets;
(iv) coating the pellets with the calcium channel blocker;
(v) optionally mixing the pellets of step (iv) with one or more pharmaceutically acceptable excipients; and
(vi) filling the pellets of step (iv) or product of step (v) into capsules.

19) A stable pharmaceutical composition comprising:
(i) 1-15% by weight calcium channel blocker,
(ii) 1-15% by weight of an ACE inhibitor
(iii) 1-10% by weight hydroxypropyl methylcellulose,
(iv) 30-90% by weight microcrystalline cellulose, and
(v) 0.1-10% by weight magnesium carbonate;
wherein the calcium channel blocker and the ACE inhibitor are not physically separated.

20) The composition according to claim 19, wherein the calcium channel blocker comprises a compound selected from the group consisting of amlodipine, felodipine, nicardipine, nifedipine, nimodipine, nisoldipine, nitrendipine, lacidipine, lercanidipine, verapamil, gallopamil, diltiazem, menthol, pharmaceutically acceptable salts thereof, and combinations thereof.

21) The composition according to claim 20, wherein the calcium channel blocker is amlodipine.

22) The composition according to claim 19, wherein the ACE inhibitor comprises a compound selected from the group consisting of ramipril, benazepril, captopril, enalapril, quinapril, perindopril, lisinopril, fosinopril, trandolapril, moexipril, pharmaceutically acceptable salts thereof, and combinations thereof.

23) The composition according to claim 22, wherein the ACE inhibitor is ramipril or benazepril.

24) The composition according to claim 19, wherein the composition has a pH of more than 6.0.

25) The composition according to claim 19, wherein the composition contains the following:

| Ingredients | Qty. (in mg) |
|---|---|
| Ramipril | 5.00 |
| Amlodipine besylate | 6.93 |
| Microcrystalline cellulose | 56.72 |
| Magnesium carbonate | 1.35 |
| Hydroxypropyl methylcellulose | 5.20 |
| (HPMC) | |
| Talc | 1.29 |
| Total | 76.50 |

26) A method for treatment of hypertension, wherein the method comprises administering a patient in need thereof a stable pharmaceutical composition according to claim 1.
